(19) United States
(12) Patent Application Publication
(10) Pub. No.: US 2003/0086987 A1
(43) Pub. Date: May 8, 2003

(54) Salvianolic acid B

(76) Inventors: Ming-shi Shiao, Taipei (TW); Tsung-Chung Lin, Cheshire, CT (US); Yuh-Lien Chen, Taipei (TW)

Correspondence Address: FISH & RICHARDSON PC, 225 FRANKLIN ST, BOSTON, MA, 02110, US

(21) Appl. No.: 10/230,542

(22) Filed: Aug. 29, 2002

Related U.S. Application Data
(60) Provisional application No. 60/316,167, filed on Aug. 30, 2001.

(51) Int. Cl. A61K 35/78; A61K 31/192

(52) U.S. Cl. 424/746; 424/757; 514/568

(57) ABSTRACT

This invention features a method for treating inflammatory injury to the cardiovascular system caused by oxidized low density lipoprotein. The method includes administering to a subject in need an effective amount of a composition containing salvianolic acid B.

### RELATED APPLICATIONS

This application claims priority to U.S. provisional application No. 60/316,167 filed on Aug. 30, 2001, the contents of which are incorporated herein by reference.

### BACKGROUND

Oxidized low density lipoprotein (oxLDL) is a key substance in the atherogenesis process. See, e.g., Ross (1993) Nature 362: 801-809. oxLDL can be generated by auto-oxidation in the presence of transition metals, by cell-mediated mechanisms, and by enzyme-mediated mechanisms. It induces early changes in the atherosclerosis process, such as the transformation of macrophages and smooth muscle cells to foam cells; the production of various pro-inflammatory cytokines and growth factors by vascular cells; and the proliferation and migration of vascular cells. See, e.g., Harada-Shiba et al. (1998) J. Biol. Chem. 273: 9681-9687. These changes consequently trigger a series of cellular responses in the arterial wall that result in the formation of atheromatous lesions. In addition, oxLDL affects the later stages of the atherosclerosis process due to its toxicity. See, e.g., Morel et al. (1984) Arteriosclerosis 4: 357-364. Accordingly, the identification of agents that modulate oxLDL could provide new therapeutics useful in treating or preventing oxLDL-induced disorders or symptoms associated with oxLDL-induced disorders.

### SUMMARY

This invention is based, in part, on use of salvianolic acid B, a compound found in Chinese herbs (e.g., Salvia miltiorrhiza), as an agent for treating or preventing oxidized low density lipoprotein induced disorders.

In one aspect, the present invention features a method for treating inflammatory injury to the cardiovascular system caused by oxidized low density lipoprotein. The method includes administering to a subject in need an effective amount of a composition containing salvianolic acid B. The inflammatory injury to the cardiovascular system may lead to atherosclerosis. The subject can be an animal or a human, e.g., a patient with inflammatory injury to the cardiovascular system caused by oxidized low density lipoprotein.

In some embodiments, the salvianolic acid B is 0.01% to 80% of the composition by weight. The composition can be a nutraceutical composition or a pharmaceutical composition. In other embodiments, the salvianolic acid B is enriched from S. miltiorrhiza.

As used herein, the term “treating” includes treatment of an existing condition, ameliorating a condition, providing palliation of a condition, or preventing the progress or development of a condition.

In another aspect, this invention features a method for treating inflammatory injury to the cardiovascular system caused by oxidized low density lipoprotein. The method includes administering to a subject in need an effective amount of a composition containing a first, a second, and a third herb extracts. The first herb extract is enriched from S. niltiorrhiza, the second herb exact is enriched from Angelica sinensis, and the third herb extract is enriched from Glycyrrhiza spp. In some embodiments, the first, the second, and the third herb extracts are in the ratio ranging from 2:2:2 to 2:0:0 (e.g., 2:1:1), respectively.

This invention also features a composition (e.g., a nutraceutical or pharmaceutical composition) comprising a first, a second, and a third herb extracts. The first herb extract is enriched from S. miltiorrhiza, the second herb exact is enriched from Angelica sinensis, and the third herb extract is enriched from Glycyrrhiza spp. In some embodiments, the first, the second, and the third are in the ratio ranging from 2:2:2 to 2:0:0 (e.g., 2:1:1), respectively. The composition can further comprise a pharmaceutically acceptable carrier.

In still another aspect, this invention features a method for treating injury to the cardiovascular system caused by physical trauma (e.g., damages to the endothelia). The method includes administering to a subject in need an effective amount of a composition containing salvianolic acid B. The injury to the cardiovascular system may lead to restenosis. In some embodiments, the salvianolic acid B is enriched from S. miltiorrhiza, and can be 0.01% to 80% of the composition (e.g., a nutraceutical or pharmaceutical composition) by weight.

Another aspect of this invention is a pharmaceutical composition that contains salvianolic acid B and a pharmaceutically acceptable carrier. The pharmaceutical composition can be used for treating the above-mentioned disorders. Salvianolic acid B in the composition can be either synthesized from organic chemicals or purified from a natural source, and can be in the form of a pharmaceutical acceptable salt. Such a salt, for example, can be formed between a negatively charged substituent (e.g., carboxylate) on salvianolic acid B and a cation. Suitable cations include, but are not limited to, sodium ion, potassium ion, magnesium ion, calcium ion, and an ammonium cation such as teteramethylammonium ion.

Other advantages or features of this invention will be apparent from the following detailed description thereof.

### DETAILED DESCRIPTION

Salvianolic acid B is found in Chinese herbs, such as the roots of S. miltiorrhiza, which includes at least 50 compounds. See, Men'shikov et al. (1952) J. Gen. Chem. 22: 1465-1467; Delorme et al. (1977) Plant Med. Phytother. 11:5-11; Ogihara et al. (1997) Bull. Coll. Sci. 64: 53-59; Lin et al. (1999) J. Nat. Prod. 62: 1500-1503; Crowley & Culvenor (1955) J. Aust. J. Chem. 8: 464-465; Li et al. (1984) Planta. Medica. 50: 227-228; Lee et al. (1987) J. Nat. Prod. 50: 157-160; and Ai et al. (1988) J. Nat. Prod. 51: 145-149. Salvianolic acid B can be enriched from the roots of S. miltiorrhiza by the methods disclosed herein. For example, one can extract salvianolic acid B from a solvent of water and ethanol. In another example, one can soak pulverized roots from S. miltiorrhiza in a sufficient volume of water to dissolve salvianolic acid B; collect the supernatant; incubate the supernatant with affinity beads (e.g., Diaion HP-20) which bind organic compounds including salvianolic acid B; and release salvianolic acid B from the beads with an eluting solvent, e.g., an aqueous solution containing 10-90% ethanol. In another example, one can remove non-water-soluble components in roots from S. miltiorrhiza by a sufficient volume of ethyl acetate; then extract salvianolic acid B with a mixture of water and ethanol (1:4, v/v) at 60° C.; and repeat the extraction step several times. The extract is combined, concentrated and is subsequently passed through a Diaion HP-20 separation column. A mixture of water and methanol is used as the developing solvent and the elutant is fractionized. Each fraction is tested by thin layer chromatography. The fractions having salvianolic acid B are pooled, and can be further applied to Sephadex LH-20 separation column, with 80% methanol as the developing solvent, to obtain a salvianolic acid B-containing product.

The purity of the thus enriched salvianolic acid B-containing product can be readily measured by any appropriate method, for example, column chromatography, or high pressure liquid chromatography analysis. The enriched product can be used as a source of salvianolic acid B to prepare a nutraceutical product. Examples of the nutraceutical products include milk or a drink (containing 0.01 to 1% by weight of salvianolic acid B), or a capsule or tablet (containing 1 to 80% by weight of salvianolic acid B). Alternatively, the enriched product can be used as a source of salvianolic acid B to prepare a pharmaceutical composition. Further, the enriched product can be used as a standard for quality control analysis.

Other extracts enriched from, e.g., Angelica sinensis, or Glycyrrhiza spp., can be prepared by methods well known in the art, such as water-ethanol extraction or a method similar to the methods described above.

Within the scope of this invention is a salvianolic acid B-containing composition itself, as well as a pharmaceutical composition that has an effective amount of salvianolic acid B for treating inflammatory injury to the cardiovascular system caused by oxidized low density lipoprotein; injury to the cardiovascular system caused by physical trauma (e.g., percutaneous transluminal coronary angioplasty, or PTCA procedure). “An effective amount” is defined as the amount of salvianolic acid B which, upon administration to a subject in need of treatment of the just-described disorders, is required to confer therapeutic effect on the treated subject. An effective amount of salvianolic acid B can range from about 10 mg/adult/day to about 1000 mg/adult/day. Effective doses will also vary, as recognized by those skilled in the art, depending on the types of disorders treated, routes of administration, excipient usage, and the possibility of co-usage with other therapeutic treatments such as use of anti-inflammatory agents.

To practice the method of the present invention, salvianolic acid B can be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A composition for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A sterile injectable composition, for example, a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

The carrier in the pharmaceutical composition must be “acceptable” in the sense of being compatible with the active ingredient of the formulation (and preferably, capable of stabilizing it) and not deleterious to the subject to be treated. For example, solubilizing agents such as cyclodextrins, which form specific, more soluble complexes with salvianolic acid B, or one or more solubilizing agents, can be utilized as pharmaceutical excipients for delivery of salvianolic acid B. Examples of other carriers include colloidal silicon dioxide, magnesium stearate, cellulose, sodium lauryl sulfate, and D&C Yellow # 10.

Without further elaboration, it is believed that the above description has adequately enabled the present invention. The following specific embodiments are, therefore, to be construed as merely illustrative, and not limitative of the remainder of the disclosure in any way whatsoever. All of the publications cited herein are hereby incorporated by reference in their entirety.

Effects of Salvianolic Acid B on oxLDL Induced-Inflammatory Injury to the Cardiovascular System

Human aortic smooth muscle cells (HASMCs, Cascade Biologics, Inc. Portland, Oreg.) were cultured in Medium 231 (Cascade Biologics, Inc.), supplemented with 5% smooth muscle growth serum (SMGS), 100 untis/mL penicillin, 100 μg/mL streptomycin, 1.25 μ/mL Fungizone (amphotericin B) at 37° C. in 5% CO₂ saturated humidity atmosphere. The medium was changed every 4 days, and cells were detached by treatment with 0.05% trypsin, 0.02% EDTA solutions. Studies were conducted on HASMCs between passages 4 and 9. Before each experiment, the medium was removed, and cells were washed and then cultured in serum-free medium containing the supplements indicated above but without SMGS.

Rescue oxLDL induced cell death HASMCs were incubated 0, 1, 1.25, 10, or 20 μg/mL salvianolic acid B (purified) in the just-described serum-free medium for 24 hr. Then, 40 μg/mL oxLDL was added to the incubated medium for another 24 hr. The cytotoxic effect can be determined using the 3-(4,5-dimethylthiazol-2-yl)-2,5-diphenyltetrazolium bromide (MTT) assay method described in Boyd (In Principle of Practice of Oncology, Devita, J. T., Hellman, S., and Rosenberg, S. A. (Eds) Vol. 3, PPO Update, No. 10, 1989). For comparison, 2.5 μg/mL probucol, demonstrated to reduce atherosclerosis, was tested as well. The data show that the cell viability of the HASMCs, after incubation with 0, 1, 1.25, 10, or 20 μg/mL salvianolic acid B and oxLDL, were 23.5% (±7.4%), 68.3% (±5.3%), 57.5% (±14.4%), 57.6% (±18.7%), and 38.3% (±18.8%), respectively. Cell viability of the probucol-treated cells is similar to that of 20 μg/mL salvianolic acid B-treated cells.

Inhibit oxLDL induced caspase-3 activation HASMCs were incubated with 20 μg/mL oxLDL for 6, 12, 24, or 48 hr, or with 1 or 10 μg/mL salvianolic acid B (purified) for 24 hr, followed by 20 μg/mL oxLDL 24 hr treatment. Cells (3×10⁵/assay) were solubilized in 5 μL phenylmethylsulfonyl fluoride and 50 μL lysis buffer (20 mM Tris, pH 7.5, 150 mM NaCl, 1 mM EDTA, 1 mM EDTA, 1% Triton X-100, 2.5 mM sodium pyrophosphate, 1 mM β-glycerophosphate, 1 mM Na₃VO₄, and 1 μg/mL Leupeptin) for 10 min at 0° C., centrifuged (15,000×g) for 10 min at 4° C. 15 μL of the protein-containing supernatant was diluted with a 6×sample buffer (125 mM Tris-HCl, pH 6.8, 4% SDS, 20% glycerol, 100 mM DTT, and 0.02% bromophenol blue) to obtain a sample solution. The sample solution was heated at 100° C. for 5 min, cooled down to room temperature, and subjected to 12% SDS-polyacrylamide gel electrophoresis. Then, proteins were transferred to PVDF membranes, blocked with 5% nonfat milk in Tris-buffered saline-Tween 20 (0.1%) at room temperature for 2 hr, and incubated overnight at 4° C. with anti-rabbit anti-caspase-3 antibody (dilution 1:1000). The membranes were washed three times in Tris-buffered saline-Tween 20, and antibody reactions were detected using horseradish peroxidase-conjugated goat anti-rabbit IgG (0.2 μg/mL) and LumiGLO chemiluminescent detection reagents (Bradford (1976) Analytical Biochem 72: 248-254).

Activated caspase-3 (17 kD) was detected in HASMCs after oxLDL treatment. However, after salvianolic acid B incubation, no activated caspase-3 was observed. The results show that salvianolic acid B inhibits oxLDL induced caspase-3 activation.

Inhibit oxLDL induced IL-1β and IL-1ra production HASMCs were incubated with 0, 1, 2.5, 10, or 20 μg/mL salvianolic acid B or 2.5 μg/mL probucol for 24 hr, followed by 20 μg/mL oxLDL 24 hr treatment. IL-1 production was determined with a sandwich ELISA using anti-IL-1 antibodies (e.g., IL-1β and IL-1ra ELISA kits from R&D Systems and Endogen, Inc. respectively). The results show that 1, 2.5, and 10 μg/mL salvianolic acid B inhibit oxLDL induced IL-1β and IL-1ra production. The data also show that IL-1β production increased after 48 hr oxLDL treatment, and reached a plateau after an additional 24 hr. Each of 1, 2.5, and 10 μg/mL salvianolic acid B doses had similar inhibitory effect on both oxLDL induced IL-1β and IL-1ra production.

The cytokine interleukin-1 (IL-1) is a key mediator in inflammatory responses (for reviews, see, e.g., Dinarello & Wolff (1993) N. Engl. J. Med. 328: 106-113). Therefore, an agent capable of mediating IL-1 production can be used for treating IL-1 related inflammatory disorders. In particular, inflammation disorders in cardiovascular system may lead to atherosclerosis and be closely correlated to more serious acute cardiovascular diseases.

A Salvianolic Acid B-Containing Composition, SAGE.

Selection of herbal components for SAGE The selection of herbal components for SAGE (Salvia miltiorrhiza, Angelica sinensis, and Glycyrrhiza uralensis Extracts) were based on the following observations and experimental results.

a. Potency in the inhibition of Cu²⁺-induced human low-density lipoprotein (LDL) oxidation;

b. Inhibition of LDL oxidation in high-glucose pretreated human LDL and LDL obtained from diabetic patients (bioactivities were observed in aqueous and ethanolic extracts of Angelica sinensis and Glycyrrhiza uralensis. In these assays, probucol (an lipophilic antioxidant and cholesterol-lowering drug), trolox (a water-soluble vitamin E analog, and aminoguanidine (an inhibitor against glycation and glycation-promoted oxidation) were used as the positive controls;

c. By comparison with various Leguminosae Chinese herbal plants in the bioassay based on the inhibition of LDL oxidation, the activity, together with low in toxicity concern, of Glycyrrhiza uralensis was high in the screening list; and

d. Toxicology concern of SAGE (Salvia miltiorrhiza, Angelica sinensis, and Glycyrrhiza uralensis Extracts) was low.

The composition of SAGE can be prepared with water-ethanol extraction of aforementioned three herbs. Further, one or more of the herb extracts can be of further purified.

Effects of SAGE on oxLDL Induced-Inflammatory Injury to the Cardiovascular System

7-month-old ApoE(−) mice were randomly divided into two groups. Animals in the control group (CT group) were fed with a diet containing 0.15% cholesterol (w/w) (a control diet). The test group was fed with the control diet containing 0.6% SAGE (extracts from S. miltiorrhiza, Angelica sinensis, and Glycyrrhiza glabra. in the ratio of 2:1:1, respectively, SAGE group). The feeding period was four months.

Aortic arch was removed from mice, and fixed in 4% paraformaldehyde in phosphate-buffered saline for 3 hr, followed by paraffin embedding, and attachment on poly-L-lysine microscope slides. Atherosclerotic plaques were observed, and the expression of IL-1βmRNA and IL-1ra mRNA in atherosclerotic plaques were examined by in situ hybridization.

Hybridization DNA probes were purchased from R&D. Tissue specimens were placed on slides, and were hybridized under identical conditions. After deparaffinization and rehydration, the slides were incubated with 0.1 M Tris-HCl and 50 mM EDTA at 37° C. for 5 min, treated with proteinase K (1 μg/mL) at 37° C. for 15 min, acetylated with 0.25% acetic anhydride in 0.1 M triethanolamine, and hybridized with the DNA probes at 42˜50° C. for 16 ˜20 hr. Finally, the slides were washed, reacted with streptavidin-alkline phosphatase, and treated with a buffer containing nitroblue tetrazolium (NBT)/5-bromo-4-chloro-3-indolylphosphate (BCIP).

In the control group, the expression IL-1βmRNA and IL-1ra was significant. While in the SAGE group, the expression of IL-1βmRNA was not statistically significant, and the expression of IL-1ra was not observed.

Effects of Salvianolic Acid B on Injury to the Cardiovascular System

New Zealand White rabbits were randomly divided into three groups. One group of rabbits was fed with a 100 g/day diet containing 2% cholesterol (a control diet, CT group). Another group was fed with the control diet containing 1.5 g/day probucol (P group), and the third group was fed with the control diet containing 375 mg/day salvianolic acid B (Sal B group). After being fed for three weeks, all rabbits underwent a femoral artery denudation. After the operation, the diet fed to each group was maintained for anther three weeks.

Evaluation of vascular histological changes was made using aortic arch, thoracic aorta, or abdominal aorta sections, which were removed from rabbits, fixed in 4% paraformaldehyde solution for 3 hr, transferred to saline, and processed for paraffin embedding. Thin sections (5 μm thick) were stained with Mayer's hematoxylin-eosin solution. LV-2 computer assisted morphometric planimetry was used to calculate intima and media areas. See, e.g., Gordon et al. (1992) Proc NatlAcad Sci USA 89: 11312-11316. In the CT group, the ratio of the intima area to the media area of the aortic arch section was 0.22 (±0.04). The ratio was about 0.1 in the P group, while the ratio was about 0.07 in the Sal B group. Further in the CT group, the ratio of the intima area to the media area of the thoracic aorta section was 0.1 (±0.02). The ratio was about 0.03 in the P group, while the ratio was about 0.02 in the Sal B group. In addition, in the CT group, the ratio of the intima area to the media area of the abdominal aorta section was 0.58 (±0.03). The ratio was about 0.33 in the P group, while the ratio was about 0.26 in the Sal B group.

Effects of Salvianolic Acid B and SAGE on Injury to the Cardiovascular System

25-week apoE (−) mice from Jackson Laboratory were randomly divided into three groups. Animals in the control group (CT group) were fed with a diet containing 0.15% cholesterol (w/w) (a control diet). Second group (SB group) was fed with the control diet containing 0.25% salvianolic acid B (w/w); and third group (SAGE group) was fed with the control diet containing 2% SAGE (w/w). The feeding period was 113 days. The results are shown in Table 1. SAGE was prepared as follows:

Salvia miltiorrhiza extract was obtained from extracts of the sliced and ground roots of Salvia miltiorrhiza by using an aqueous ethanol solution (water: ethanol =4:1, v/v). The weight to volume ration of Salvia miltiorrhiza to extraction solvent was 1:10 (w/v).

Angelica sinensis extract was obtained from ethanolic extracts (1:10, w/v).

Glycyrrhizae radix (Glycyrrhiza uralensis) extract was obtained from ethanolic extracts (1:10, w/v).

The weight ratio of Salvia miltiorrhiza extract, Angelica sinensis extract, and Glycyrrhiza uralensis extract was 2:1:1, respectively, based on the dried weights of the raw herbs used.

TABLE 1
Atherosclerotic area in the apoE (−) mice

| Area | CT (n = 4) | SB (n = 6) | SAGE (n = 6) |
|---|---|---|---|
| Media (10³ μm²) | 20,581 ± 18,343 | 9,763 ± 2,239 | 10,900 ± 3,224 |
| Intima(10³ μm²) | 21,056 ± 29,427 | 1,563 ± 2,504 | 2,129 ± 2,588 |
| Intima/Media (ratio) | 0.770 ± 0.490 | 0.128 ± 0.170 | 0.167 ± 0.143 |
| P value | — | 0.0164 | 0.0195 |
| Inhibition (%) | | 83 | 78 |

1. The Intima/Media ratio was calculated from every aortic slices of each animal in the same group. It was not obtained from the dividing of mean value of intima thickness by that of media of the entire group. Results were shown as mean ± S.D.
2. Inhibition (%) of atherosclerosis was compared with control group.

### OTHER EMBODIMENTS

All of the features disclosed in this specification may be combined in any combination. Each feature disclosed in this specification may be replace by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to salvianolic acid B also can be made and used to practice this invention. Thus, other embodiments are also within the claims.

What is claimed is:

1. A method for treating inflammatory injury to the cardiovascular system caused by oxidized low density lipoprotein, comprising administering to a subject in need thereof an effective amount of a composition containing salvianolic acid B.

2. The method of claim 1, wherein the inflammatory injury to the cardiovascular system leads to atherosclerosis.

3. The method of claim 1, wherein the salvianolic acid B is 0.01% to 80% of the composition by weight.

4. The method of claim 1, wherein the salvianolic acid B is enriched from Salvia miltiorrhiza.

5. The method of claim 4, wherein the salvianolic acid B is 0.01% to 80% of the composition by weight.

6. A method for treating inflammatory injury to the cardiovascular system caused by oxidized low density lipoprotein, comprising administering to a subject in need thereof an effective amount of a composition containing a first, a second, and a third herb extracts, wherein the first extract is enriched from Salvia miltiorrhiza, the second extract is enriched from Angelica sinensis, and the third extract is enriched from Glycyrrhiza spp.

7. The method of claim 6, wherein the inflammatory injury to the cardiovascular system leads to atherosclerosis.

8. The method of claim 6, wherein the first, second, and the third extracts are in a ratio ranging from 2:0:0 to 2:2:2, respectively.

9. The method of claim 8, wherein the first, second, and the third extracts are in a ratio of 2:1:1, respectively.

10. The method of claim 6, wherein the salvianolic acid B is 0.01% to 80% of the composition by weight.

11. The method of claim 6, wherein the salvianolic acid B is enriched from Salvia miltiorrhiza.

12. The method of claim 11, wherein the salvianolic acid B is 0.01% to 80% of the composition by weight.

13. A method for treating injury to the cardiovascular system caused by physical trauma, comprising administering to a subject in need thereof an effective amount of a composition containing salvianolic acid B.

14. The method of claim 13, wherein the injury to the cardiovascular system leads to restenosis.

15. The method of claim 13, wherein the salvianolic acid,B is 0.01% to 80% of the composition by weight.

16. The method of claim 13, wherein the salvianolic acid B is enriched from Salvia miltiorrhiza.

17. The method of claim 16, wherein the salvianolic acid B is 0.01% to 80% of the composition by weight.
